(19) United States
(12) Patent Application Publication
(10) Pub. No.: US 2024/0181104 A1
(43) Pub. Date: Jun. 6, 2024

(54) METHODS AND SYSTEMS FOR REMOVING HYDROGEN PEROXIDE FROM A GAS

(71) Applicant: ENTEGRIS, INC., Billerica, MA (US)

(72) Inventors: Jürgen Michael Lobert, Franklin, MA (US); Joseph Wildgoose, North Smithfield, RI (US); Reena Srivastava, Franklin, MA (US); Edward John Washington, Wrentham, MA (US)

(21) Appl. No.: 18/527,045

(22) Filed: Dec. 1, 2023

Related U.S. Application Data
(60) Provisional application No. 63/429,428, filed on Dec. 1, 2022.

(51) Int. Cl. A61L 2/20; A24B 15/18; A61L 2/00; B01D 53/46; B01D 53/81; B01J 20/04; B01J 20/20; B01J 20/28

(52) U.S. Cl. CPC A61L 2/208; A24B 15/183; A61L 2/0094; B01D 53/46; B01D 53/81; B01J 20/041; B01J 20/046; B01J 20/20; B01J 20/2804; A61L 2202/122; A61L 2202/123; A61L 2202/13; A61L 2202/21; A61L 2202/24; A61L 2202/25; B01D 2253/102; B01D 2253/25; B01D 2253/34

(57) ABSTRACT

Described are processes and equipment that are useful to remove hydrogen peroxide from a gas, for example from a hospital room as a step of sterilizing the room using hydrogen peroxide, or from a flow of exhaust air that is produced by sterilization equipment that uses hydrogen peroxide as a sterilizing agent.

### FIELD

This description relates to processes and equipment that are useful to remove hydrogen peroxide from a gas.

### BACKGROUND

Hydrogen peroxide is known to be useful as a sterilizing agent in both liquid and gaseous forms. Hydrogen peroxide is a potent oxidizer that effectively kills many different bacteria, microbes, spores, fungi, etc., such as those that are present in biomass (e.g., cannabis), on medical equipment and items, and the like.

Hydrogen peroxide sterilization, also known as “hydrogen peroxide gas sterilization” or “hydrogen peroxide vapor sterilization,” is a low temperature sterilization process commonly used to sterilize heat-sensitive items. A hydrogen peroxide sterilization process involves contacting items to be sterilized with hydrogen peroxide vapor, within a sterilization environment, and allowing the hydrogen peroxide sufficient time to contact and kill or inactivate biologically active materials at surfaces of the items. A hydrogen peroxide sterilization cycle typically requires less time than alternative forms of sterilization such as ethylene oxide sterilization or manual wipe-downs.

Because hydrogen peroxide is a common household item, users consider it to be a non-toxic and environmentally safe sterilizing agent. However, when used for industrial level sterilization, aqueous concentrations are up to 90% and can cause health concerns when evaporated. The OSHA 8-hour Time Weighted Average (TWA), permissible exposure limit (PEL) for hydrogen peroxide in air as per US government regulation 29 CFR 1910.1000 is 1 part per million (ppm).

The healthcare industry uses hydrogen peroxide gas sterilization methods for sterilizing heat-sensitive items at low temperature. Examples include medical devices, implants, and instruments that contain plastic such as wound dressings, stents, catheters, packaged products, and the like. Hydrogen peroxide is also used for sterilizing hospital rooms by flooding the room interior with gaseous hydrogen peroxide. The amount of hydrogen peroxide in the room must be brought to an acceptable level before the room can be occupied.

Hydrogen peroxide sterilization is also used for sterilizing temperature-sensitive items such as plants, e.g., cannabis, that contain valued chemical molecules that have specific metabolic or chemical effects, without degrading those molecules. Equipment that is designed for performing methods of sterilizing cannabis using hydrogen peroxide vapor is commercially available.

### SUMMARY

Described as follows, generally, are methods and systems that are useful to reduce an amount of hydrogen peroxide that is contained in a gas, using carbon adsorbent that is treated to be particularly effective for eliminating the presence of hydrogen peroxide. The gas may be any gas that contains an amount of hydrogen peroxide that is desirably removed. According to a particular example the gas may be an atmosphere of air that is contained in an enclosed space, such as a hospital room that has been sterilized by using hydrogen peroxide as a sterilizing agent. In a different example, the gas may be a flow of exhaust air that is produced by sterilization equipment that uses hydrogen peroxide as a sterilizing agent.

Hydrogen peroxide is an irritant for the human respiratory system. When hydrogen peroxide vapor is used in a sterilization method, the method produces air that contains hydrogen peroxide vapor. In specific, when hydrogen peroxide is used to sterilize a hospital room, the room is flooded with hydrogen peroxide vapor. The hydrogen peroxide must eventually be removed from the air atmosphere of the room, typically by circulating the air in the room through a filter that is placed in the room.

When hydrogen peroxide is used in sterilization equipment that includes a sterilization chamber, items to be sterilized are placed in the sterilization chamber and exposed to hydrogen peroxide. After completing a sterilization step, the sterilization chamber atmosphere, which contains concentrated hydrogen peroxide, is then exhausted into the atmosphere of the space that contains the sterilization equipment. When the sterilization equipment is in an enclosed facility, and when multiple sterilization systems are operated in the enclosed facility, the amount of hydrogen peroxide that is added to the facility atmosphere from the exhaust air can potentially accumulate to levels that exceed established health limits.

Methods and equipment of the present description can be useful to reduce or substantially eliminate the presence of hydrogen peroxide in a gas that contains hydrogen peroxide, e.g., from an air atmosphere of a hospital room following sterilization with hydrogen peroxide, or from exhaust air produced by a hydrogen peroxide vapor sterilization process. As described, the gas is contacted with carbon adsorbent that has been treated with a combination of a caustic agent (e.g., a strong base) and a reducing agent. Contacting the gas with the treated carbon adsorbent can remove much or substantially all of the hydrogen peroxide that is contained in the gas.

Useful examples of treated carbon adsorbent can contain potassium hydroxide (KOH) as a caustic agent and potassium iodide (KI) as a reducing agent. The caustic agent at the surface and the reducing agent at the surface can both be chemically capable of interacting with hydrogen peroxide vapor in a manner that converts the hydrogen peroxide to different and less toxic compounds such as oxygen or water. The carbon surface of the adsorbent may also interact with the hydrogen peroxide, e.g., by a catalytic mechanism, to convert the hydrogen peroxide to a less toxic compound such oxygen or water.

In one aspect, the following description relates to a method of processing a gas to remove hydrogen peroxide from the gas. The method includes: providing gas that contains hydrogen peroxide, contacting the gas with carbon adsorbent that includes caustic agent and reducing agent, to reduce a concentration of the hydrogen peroxide in the gas.

In another aspect, the following description relates to a system for processing a gas that contains hydrogen peroxide to remove hydrogen peroxide from the gas. The system includes: a housing that includes an inlet, an outlet, and an interior between the inlet and the outlet; and carbon adsorbent that comprises porous carbon adsorbent base treated with caustic agent and reducing agent, at the interior, between the inlet and the outlet.

In another aspect, the description relates to a method of sterilizing cannabis. The method includes: placing cannabis in a sterilization chamber that contains an atmosphere that includes air; dispensing hydrogen peroxide vapor into the air atmosphere in the sterilization chamber; for a period of time, allowing the hydrogen peroxide to inactivate biologically active materials contained in the cannabis; and after the period of time, removing the air from the sterilization chamber as exhaust air and contacting the exhaust air with carbon adsorbent that includes caustic agent and reducing agent to reduce a concentration of the hydrogen peroxide in the exhaust air.

In another aspect, the present description relates to a method of sterilizing a hospital room. The method includes: dispensing hydrogen peroxide vapor into an air atmosphere within the room; allowing the hydrogen peroxide to contact surfaces in the room to sterilize the surfaces; and contacting the air atmosphere with carbon adsorbent that includes caustic agent and reducing agent to allow hydrogen peroxide to contact surfaces of the carbon adsorbent to reduce a concentration of the hydrogen peroxide in the air atmosphere.

### BRIEF DESCRIPTION OF THE FIGURES

FIGS. 1A, 1B, 1C, and 1D illustrate an example filter apparatus as described.

FIGS. 2A and 2B illustrate an example of a transition plate component of an example filter apparatus. Mounting hardware for, e.g., suspended installation, may be added and is not shown.

FIGS. 3A, 3B, and 3C, illustrate an example filter apparatus as described, including an installed transition plate.

FIG. 4 shows an example sterilization system with a filter as described.

FIG. 5 shows an alternate example sterilization system with a filter as described.

The figures are schematic and not necessarily to scale.

### DETAILED DESCRIPTION

Described are novel adsorbents, processes, and equipment that can be used to remove hydrogen peroxide from a gas, such as air, by contacting the gas with carbon adsorbent that has been treated to add chemical materials to the surface of the adsorbent that destroy the hydrogen peroxide, e.g., react with the hydrogen peroxide and convert the hydrogen peroxide to reaction products that have a lower toxicity than the hydrogen peroxide, such as water and oxygen.

Useful carbon adsorbents may be treated with a combination of a caustic agent (e.g., strong base) and a reducing agent. Contacting the gas with the treated carbon adsorbent removes hydrogen peroxide from the gas and reduces the concentration of hydrogen peroxide in the gas.

The gas, e.g., air, that is being processed to remove hydrogen peroxide may be any gas that contains an amount of hydrogen peroxide that is desirably removed from the gas. In a particular example, the gas is a flow of effluent air or “exhaust air” that is produced by sterilization equipment that is used to perform hydrogen peroxide vapor sterilization, meaning a process of contacting hydrogen peroxide vapor with items to be sterilized, typically within an enclosed sterilization chamber. In other examples, the air may be an atmosphere contained in an enclosed space such as a hospital room that contains hydrogen peroxide vapor that was introduced into the space for the purpose of sterilizing the space.

The air contains an amount of hydrogen peroxide vapor that is desirably removed. The air may contain typical constituents of dry air (approximately 78 percent nitrogen, 21 percent oxygen, and about 0.9 percent argon and 0.04 percent carbon dioxide), in addition to optional water vapor, many trace gases, and, per the present description, a concentration of hydrogen peroxide vapor at, for example, less than 10,000, less than 1,000, or less than 100 parts per million (ppm).

An amount of a hydrogen peroxide in a volume of air may be described as a percentage, or alternately in terms of parts per million or parts per billion. The terms “parts per million” and “parts per billion” are used herein in a manner that is consistent with the use of these terms in the chemical arts. In this respect, parts per million (“ppm”) is commonly used as a measure of small levels (concentrations) of an impurity in a gas, expressed as one part of contaminant per one million parts of air, either on a molecular or volume basis. One part per million is equal to 1×10⁻⁶ or 0.0001 percent of a total substance. One part per billion (“ppb”) is equal to 1×10⁻⁹ or 0.0000001 percent of a total substance.

According to methods as described, the gas (e.g., air) can be passed over surfaces of carbon adsorbent that is treated with caustic agent and reducing agent, to cause the gas and hydrogen peroxide contained in the gas to contact the carbon adsorbent. When the hydrogen peroxide contacts the treated surface of the adsorbent, the hydrogen peroxide is converted to less toxic chemical compounds such as water and oxygen.

A carbon adsorbent is a concentrated carbon material that is derived from carbon-containing polymeric materials or from carbon-based materials having a natural source. Examples include: carbon formed by pyrolysis of synthetic hydrocarbon resins such as polyacrylonitrile, sulfonated polystryrene-divinylbenzene, polyvinylidene chloride, etc.; cellulosic char; charcoal; and activated carbon formed from natural source materials such as coconut shells, pitch, wood, petroleum, coal, etc. Activation of a carbon (to form “activated carbon”) means that the porosity has been altered, for example, by steam treatment, to maximize the uptake of gases.

A useful carbon adsorbent may have any suitable form, such as a form of granules (also referred to as “particles”). Granules are individual pieces of carbon adsorbent, each piece having a relatively small size, such as less than 2 centimeters, or less than 1 or 0.5 centimeter, e.g., particles that pass through a mesh having a mesh size of 50 to 20, which corresponds to particles of 0.3 to 0.9 millimeter. The particles may have any range of useful particle size or shape. Example shapes include beads, granules, pellets, fibers, tablets, shells, saddles, powders, irregularly-shaped particulates, extrudates of any shape and size, cloth or web form materials, and composites (of the adsorbent with other components), as well as comminuted or crushed forms of the foregoing types of adsorbent materials.

A carbon adsorbent as described is treated with a useful amount of caustic agent, a base, e.g., a strong base such as potassium hydroxide (KOH), and with a useful amount of reducing agent such as potassium iodide (KI), to place these compounds or their ionic constituents at surfaces of the carbon adsorbent. According to examples, the caustic agent (e.g., KOH) may be applied to the adsorbent in an amount that is less than 5 weight percent caustic agent based on the weight of the adsorbent, e.g., in an amount in a range from 1 to 3 weight percent. The reducing agent (e.g., KI) may also be applied to the adsorbent in an amount that is less than 5 weight percent reducing agent based on the weight of the adsorbent, e.g., in an amount in a range from 1 to 4 weight percent.

The caustic agent and the reducing agent may be applied to the carbon adsorbent surface by methods that are known to be useful to apply these materials or their derivative ions to a surface of carbon adsorbent. As applied, the caustic agent or the reducing agent, or both, may be present partially or entirely in an ionic form, meaning, with reference to the examples of potassium hydroxide and potassium iodide, the surface will contain potassium ions (K⁺), hydroxide ions (OH⁻), and iodine ions (I⁻). Methods of applying chemical materials to carbon adsorbent surfaces are known, with examples being described in U.S. Pat. No. 9,517,445 and United States Patent Publication 2002/0152579.

A device that will be useful for performing a method as described can be in the form of a filtration apparatus that contains a housing that defines a chamber at an interior of the housing, an inlet that passes from an exterior to an interior of the housing, an outlet that passes from the interior to an exterior of the housing, and carbon adsorbent treated with caustic agent and reducing agent at the interior. A gas that enters the housing at the inlet passes through the interior, contacts the carbon adsorbent at the interior, and then can exit the housing through the outlet.

The inlet is adapted to receive a flow of gas that contains hydrogen peroxide to be removed from the gas by passing the gas through the carbon adsorbent. For use to remove hydrogen peroxide from an atmosphere of an enclosed space such as a hospital room after the room has been sterilized using hydrogen peroxide, the inlet may be an opening that is adapted to receive air taken from the enclosed space. The air may be static air in the space, and may be directed through the inlet opening using a fan or other flow-directing device that is located at the inlet as part of the filtration apparatus, or instead as a separate device. The air is brought into the filtration apparatus through the inlet, passes through the treated carbon adsorbent, and then passes from the filtration apparatus through the outlet with a reduced concentration of hydrogen peroxide.

According to a different method, the inlet of a filtration apparatus is adapted to receive a flow of effluent gas from an upstream process apparatus (e.g., a sterilization apparatus) that produces the effluent gas (e.g., air), which contains an amount of hydrogen peroxide vapor. The inlet may be adapted to receive a single flow of gas from a single upstream process apparatus, or may be adapted to receive multiple separate flows of gas from two or more separate upstream process apparatuses, each of which produces a flow of effluent gas that contains hydrogen peroxide. The flow of effluent gas from the process apparatus may be under sufficient pressure to cause the gas to flow from the process apparatus and through the inlet and filter of the filtration apparatus. Optionally, the gas may be propelled under pressure from the upstream process device to the filtration device by a fan that is part of the process apparatus, that is part of the filtration apparatus, or that is separate from both.

The carbon adsorbent may be held and supported at the interior of a filtration apparatus in any useful manner, for example as part of a sheet that contains two porous membranes arranged in a layered fashion to support a layer of activated carbon particles between the two sheets. Gas (e.g., air) may flow through the sheet, through the two porous membranes of the sheet, with the gas passing through the carbon adsorbent particles to contact the particle surfaces. The sheet may be folded or pleated, etc., to place a high surface area of the sheet within the filtration apparatus between the inlet and the outlet.

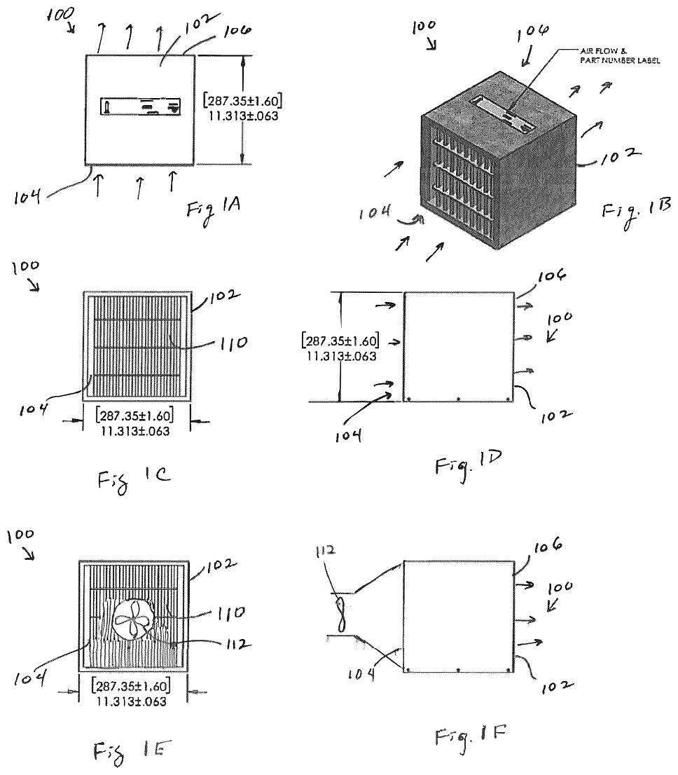

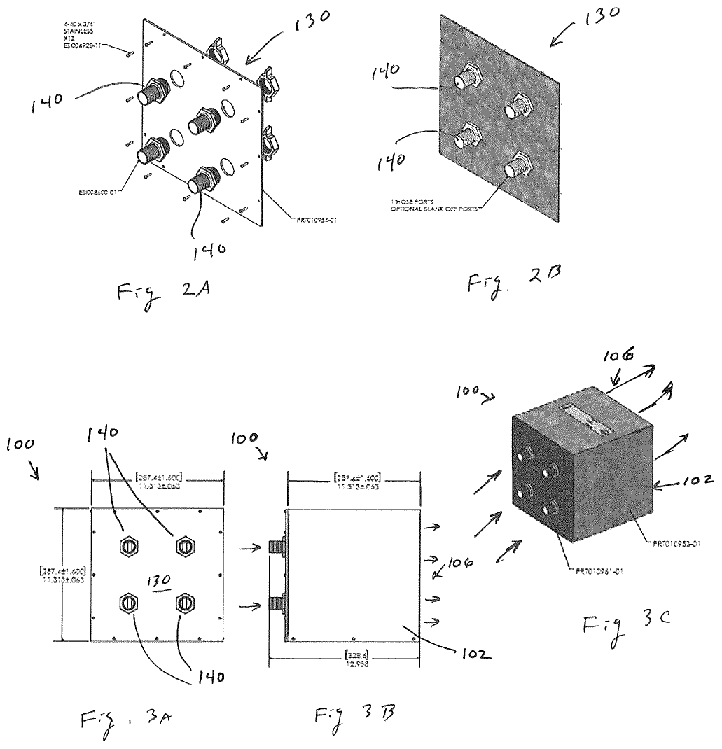

FIGS. 1A, 1B, 1C, and 1D show an example filtration apparatus. FIG. 1A is a top view of apparatus 100, FIG. 1B is a top perspective view of the apparatus, FIG. 1C is a front view looking at inlet 104, and FIG. 1D is a side view. As shown, apparatus 100 includes housing 102 which has a front or front opening 104, which is open as illustrated, without a cover (e.g., plate) over the front opening. The apparatus also includes a back or back opening 106, and an interior that contains pleated filter 110 that contains treated carbon adsorbent as described herein. A flow of gas (see arrows) can enter the housing interior through front opening 104, pass through filter 110 located at the interior, and the exit the housing interior by passing out of back opening 106. Gas that flows into the interior has a concentration of hydrogen peroxide desirably to be reduced. Upon contacting the carbon adsorbent, the hydrogen peroxide is converted to reaction products such as water and oxygen, and the concentration of hydrogen peroxide in the gas is significantly reduced before the gas passes through back opening 106 to exit housing 102.

Housing 102 may be of a size and form factor that will be useful to contain a filter of a size and flow capacity to handle movement of any particular volume and rate of gas. The dimensions may be as needed, with the illustrated housing having exterior dimensions nominally (approximately) of one foot by one foot by one foot, approximately 300×300×300 mm. The housing may be adapted to be portable, moveable, adapted to be placed at a floor or moveable support, or mounted to a ceiling or wall or support at a desired height.

The example filtration apparatus 100 may also include one or more fans as part of the apparatus, to produce a flow of air through the filter. Referring to FIGS. 1E and 1F, example filtration apparatus 100 includes one or more fans 112 located at front opening 104, which can be used to produce a flow of air through front opening 104, the air then passing through filter 110 and then exiting housing 102 through back opening 106.

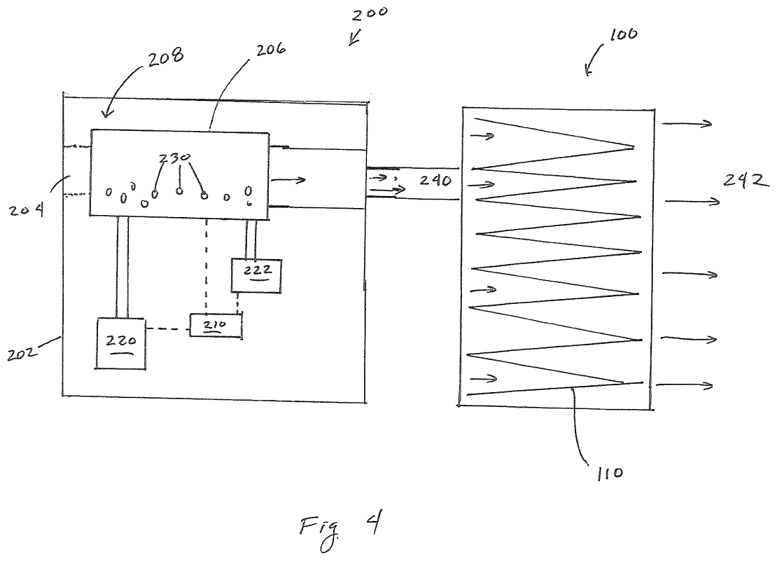

A housing 102 as illustrated at FIGS. 1A through 1D can be adapted to receive a flow of gas from a single gas source, or to receive multiple separate flows of gases from multiple different gas sources. As shown at FIGS. 2A and 2B, a transition plate 130 may be adapted to fit over front opening 104 to cover and seal the opening. The plate may include one or more adapters 140 that individually attach one or more flow conduits (not shown) to housing 102, to allow each flow conduit to direct a flow of gas into the interior of housing 102 to contact filter 110. Each flow of gas may come from a processing apparatus such as a hydrogen peroxide sterilization chamber.

In more detail, referring to FIGS. 2A (un-assembled view) and 2B (assembled view), transition plate 130 is sized to cover and seal front opening 104 of housing 102 of FIGS. 1A through 1D. Plate 130 includes multiple (four, as illustrated) adapters 140 that pass through openings in plate 130 and attach mechanically to plate 130. As illustrated, each adapter 140 includes a barbed surface that is designed to engage the end of a conduit (e.g., a hose or a pipe), to attach the conduit to plate 130 and housing 102, after which gas can flow through the conduit, through plate 130, and into housing 120 to pass through filter 110 and exit housing 102 through back opening 106. Other mechanical attachment types may be used to attach adapter 140 to a conduit, instead of barbed surfaces, e.g., a tube fitting, valve, orquick connector.

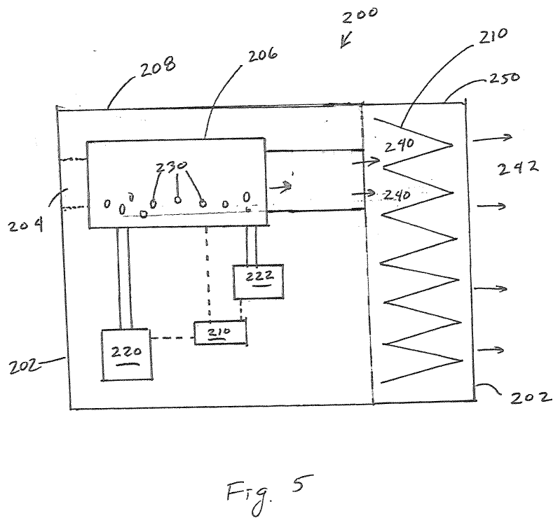

Referring to FIGS. 3A, 3B, and 3C, plate 130 is mounted to cover front opening 104 of housing 102 of FIGS. 1A through 1D. Plate 130 may attach to housing 102 in any manner, such as by screw fasteners at a perimeter of plate 130, or by any form of latch, fastener, or friction-fit engagement that may allow for faster dis-engagement and engagement of plate 130 to housing 102.

By a specific use of the present methods and filtering systems, exhaust air from a hydrogen peroxide vapor sterilization apparatus can be processed to remove hydrogen peroxide from the exhaust air. The sterilization apparatus may be used to sterilize any types of items or materials, such as a medical item, biomass (e.g., cannabis or other plant material), or a different material or product. The sterilization apparatus produces exhaust air that contains a concentration of hydrogen peroxide. The exhaust air is passed through a filtration apparatus as described herein, and the concentration of hydrogen peroxide in the exhaust air is reduced.

In an even more specific application, the exhaust air is produced by a hydrogen peroxide vapor sterilization apparatus that processes biomass such as cannabis, to sterilize the cannabis. An example of such an apparatus and method is shown at FIG. 4.

At FIG. 4, hydrogen peroxide vapor sterilization apparatus 200 includes housing 202, interior 208, and front opening 204, which provides access to an interior of sterilization chamber 206. Apparatus 200 also includes a source 220 of hydrogen peroxide vapor, vacuum system 222, control system 210, and (not shown) various flow control and condition control devices, and sensors such as timers, thermostats, temperature and pressure monitors, which together monitor and control conditions of the sterilization chamber and sterilization process.

As illustrated, sterilization chamber 206 contains biomass 230, which may be cannabis or another plant material that contains biologically active molecules or agents that are desirably deactivated. To sterilize the biomass and inactivate undesired biologically active contaminants such as fungi, bacteria, or microbes, etc., hydrogen peroxide vapor is generated at source 220 and is dispensed into sterilization chamber 206, which encloses biomass 230. The hydrogen peroxide vapor is allowed to contact the biomass and penetrate the biomass, and remain in contact with the biomass for an amount of time that is effective to sterilize the biomass and inactivate the undesired biologically active contaminants.

The atmosphere in the sterilization chamber begins as air, and the hydrogen peroxide vapor is added to the chamber and the initial air atmosphere. Hydrogen peroxide can be added to the air atmosphere in the sterilization chamber to achieve a concentration of hydrogen peroxide that is useful for sterilization.

The sterilization process can be performed at ambient temperature (e.g., from 15 to 40 degrees Celsius, or from 20 to 25 degrees Celsius) and at ambient pressure (e.g., from 12 to 15 pounds per square inch (gauge) (from 900-1200 kPA).

After a time period that is useful to allow the hydrogen peroxide to effectively sterilize the biomass, the gaseous atmosphere from the sterilization chamber is caused to flow from apparatus 200 to filtration apparatus 100, which contains filter 110 that includes carbon adsorbent treated with caustic agent and reducing agent.

Exhaust air 240 flows from sterilization apparatus 200 into filtration apparatus 100, passes through filter 110, and then exits filtration apparatus 100 as filtered air 242. Hydrogen peroxide that is contained in exhaust air 240 contacts carbon adsorbent of filter 110 at the interior of apparatus 100, and hydrogen peroxide contained in exhaust air 240 is converted to compounds such as water and oxygen. The filtered exhaust air then exits apparatus 100 as filtered exhaust air 242, containing a concentration of hydrogen peroxide that is significantly reduced compared to the concentration of hydrogen peroxide within exhaust air 240, e.g., the concentration of hydrogen peroxide contained in filtered exhaust air 242 may be less than 50 percent, or less than 20, 10, 5, or percent of the concentration of hydrogen peroxide contained in exhaust air 240.

Examples of useful low temperature sterilization systems adapted to sterilize bulk cannabis and other biomass, are available commercially.

According to examples of useful apparatuses as described, a sterilization system may include a sterilization apparatus that is attached to a separate filtration apparatus as shown at FIG. 4. The sterilization system 200 is a separate unit that can be attached and detached from the filter system 100 by a conduit as illustrated.

According to alternate systems, a filtration apparatus of the present description can be incorporated into a sterilization apparatus, e.g., integrated into the sterilization apparatus by including the filter in a single housing of a sterilization apparatus. As illustrated at FIG. 5, a sterilization apparatus 200 includes components of the example apparatus of FIG. 4, with filter 210 located at an interior of housing 202. Using this apparatus, exhaust air 240 passes from sterilization chamber 206, through filter 242, and into filter chamber 250, which contains filter 110, containing adsorbent that includes caustic agent and reducing agent. Filter chamber 250 and filter 110 are located within housing 202 of sterilization apparatus 200. Exhaust air 240 passes through filter 110, and the exits filter chamber 250 and apparatus 200 as filtered exhaust air 242.

According to a different use of the present methods and filtering systems, air that is contained in an environment of an enclosed space such a room, e.g., a hospital room, that contains hydrogen peroxide vapor, can be processed using a filtration apparatus as described herein, for example as illustrated at FIGS. 1E and 1F, to remove hydrogen peroxide from the air.

According to an example of such a use, an enclosed room is sterilized by dispensing hydrogen peroxide vapor into the room and allowing the hydrogen peroxide vapor to contact surfaces, including by penetrating porous or fibrous surfaces in the room. The hydrogen peroxide vapor is held in the room for a period of time during which the hydrogen peroxide will effectively sterilize the room.

After the room is sterilized, the unused hydrogen peroxide vapor must be removed from the air environment in the room. A filtration apparatus such as exemplary filtration apparatus 100 of FIGS. 1D and 1E may be used to circulate air of the room, containing hydrogen peroxide, through a filter that contains treated carbon adsorbent as described herein, to remove the hydrogen peroxide from the room. The air may be static air (“bulk” air) that is not significantly circulating with the room. To cause the air to be circulated through the filter, for removing the hydrogen peroxide from the air, apparatus 100, including fans 112, can be placed in the room, e.g., on a floor or supported vertically by a table, carrier, or the like. Fans 112 can be powered on to cause air from the room to be drawn into front opening 104, to then pass through filter 110, which removes hydrogen peroxide from the air. The filtered air (filtrate) passes from filter 110 and through back opening 106. The filtrate that exits back opening 106 has a lower concentration of hydrogen peroxide compared to the concentration of hydrogen peroxide in the air that enters front opening 104.

1. A method of processing a gas to remove hydrogen peroxide from the gas, the method comprising:
providing gas that contains hydrogen peroxide,
contacting the gas with carbon adsorbent that includes caustic agent and reducing agent, to reduce a concentration of the hydrogen peroxide in the gas.

2. The method of claim 1, wherein the caustic agent comprises potassium hydroxide, and the reducing agent comprises potassium iodide.

3. The method of claim 1, wherein the adsorbent contains up to 5 weight percent caustic agent based on weight carbon adsorbent.

4. The method of claim 1, wherein the adsorbent contains from up to 5 weight percent reducing agent based on weight carbon adsorbent.

5. The method of claim 1, comprising reducing the concentration of hydrogen peroxide by at least 50 percent.

6. The method of claim 5, wherein the concentration of hydrogen peroxide is below 1 part per million.

7. The method of claim 1, wherein the gas passing into the carbon adsorbent has a temperature below 40 degrees Celsius.

8. The method of claim 1, wherein the gas passing into the carbon adsorbent has a pressure in a range from 12 to 15 pounds per square inch (gauge) (900-1200 kilopascal (kPA)).

9. The method of claim 1, wherein the gas is exhaust air of a sterilization chamber attached to the inlet.

10. The method of claim 9, wherein the exhaust air is provided by two or more sterilization chambers attached to the inlet.

11. The method of claim 9, wherein the sterilization chamber performs a sterilization method that comprises:
Placing an item to be sterilized in the sterilization chamber,
closing the sterilization chamber,
dispensing hydrogen peroxide into the sterilization chamber.

12. The method of claim 11, wherein the item comprises cannabis.

13. The method of claim 11, wherein the item is a medical device or a medical instrument.

14. The method of claim 1, wherein the gas is static air at an interior of a hospital room, the method comprising:
dispensing hydrogen peroxide vapor into an air atmosphere within the room,
allowing the hydrogen peroxide to contact surfaces in the room to sterilize the surfaces, and
contacting the air atmosphere with the carbon adsorbent to allow the air atmosphere to contact the carbon adsorbent and reduce a concentration of hydrogen peroxide in the air atmosphere.

15. A system for processing a gas that contains hydrogen peroxide to remove hydrogen peroxide from the gas, the system comprising:
a housing that includes an inlet, an outlet, and an interior between the inlet and the outlet,
at the interior, carbon adsorbent that comprises porous carbon adsorbent base treated with caustic agent and reducing agent, between the inlet and the outlet.

16. The system of claim 15, wherein the caustic agent comprises potassium hydroxide and the reducing agent comprises potassium iodide.

17. The system of claim 15, wherein the adsorbent contains up to 5 weight percent caustic agent based on weight carbon adsorbent.

18. The system of claim 15, wherein the adsorbent contains from up to 5 weight percent reducing agent based on weight carbon adsorbent.

19. The system of claim 15, comprising a pleated filter membrane that contains the carbon adsorbent.

20. The system of claim 15, comprising a sterilization apparatus attached to the inlet, the sterilization apparatus adapted to produce exhaust air that contains hydrogen peroxide.

21. The system of claim 15, comprising two or more sterilization apparatuses attached to the inlet, each sterilization apparatus adapted to produce exhaust air that contains hydrogen peroxide.

22. The system of claim 15, wherein the exhaust air contains less than 10,000 parts per million hydrogen peroxide.

23. A method of sterilizing cannabis, the method comprising:
placing cannabis in a sterilization chamber that contains an atmosphere that includes air,
dispensing hydrogen peroxide vapor into the air atmosphere in the sterilization chamber,
for a period of time, allowing the hydrogen peroxide to inactivate biologically active materials contained in the cannabis,
after the period of time, removing the air from the sterilization chamber as exhaust air and contacting the exhaust air with carbon adsorbent that includes caustic agent and reducing agent to reduce a concentration of the hydrogen peroxide in the exhaust air.

24. The method of claim 23, wherein the caustic agent comprises potassium hydroxide, and the reducing agent comprises potassium iodide.

25. The method of claim 23, wherein the adsorbent contains up to 5 weight percent caustic agent based on weight carbon adsorbent.

26. The method of claim 23, wherein the adsorbent contains from up to 5 weight percent reducing agent based on weight carbon adsorbent.

27. The method of claim 23, wherein the exhaust air contains less than 10,000 parts per million hydrogen peroxide.

28. The method of claim 23, comprising reducing the concentration of hydrogen peroxide by at least 50 percent.

29. The method of claim 23, wherein the cannabis, the air atmosphere, and the exhaust air passing into the carbon adsorbent are at a temperature below 40 degrees Celsius.

30. The method of claim 23, wherein the exhaust air passing into the carbon adsorbent has a pressure in a range from 12 to 15 pounds per square inch (gauge) (900-1200 kilopascal (kPA)).

31. A method of sterilizing a hospital room, method comprising:
dispensing hydrogen peroxide vapor into an air atmosphere within the room,
allowing the hydrogen peroxide to contact surfaces in the room to sterilize the surfaces, and
contacting the air atmosphere with carbon adsorbent that includes caustic agent and reducing agent, to allow hydrogen peroxide to contact surfaces of the carbon adsorbent to reduce a concentration of the hydrogen peroxide in the air atmosphere.

32. The method of claim 31, wherein the caustic agent comprises potassium hydroxide, and the reducing agent comprises potassium iodide.

33. The method of claim 31, wherein the adsorbent contains up to 5 weight percent caustic agent based on weight carbon adsorbent.

34. The method of claim 31, comprising reducing the concentration of hydrogen peroxide by at least 50 percent.
